(19) United States
(12) Patent Application Publication
(10) Pub. No.: US 2013/0041244 A1
(43) Pub. Date: Feb. 14, 2013

(54) IMPLANTABLE DEVICE FOR DETECTING A VESSEL WALL EXPANSION

(71) Applicants: Peter WOIAS, Freiburg (DE); Martin Zens, Freiburg (DE); Philipp Bingger, Freiburg (DE)

(72) Inventors: Peter WOIAS, Freiburg (DE); Martin Zens, Freiburg (DE); Philipp Bingger, Freiburg (DE)

(21) Appl. No.: 13/603,639

(22) Filed: Sep. 5, 2012

(30) Foreign Application Priority Data
Mar. 5, 2010 (DE) 10 2010 010 348.9
Feb. 28, 2011 (EP) PCT/EP2011/000974

(51) Int. Cl. A61B 5/053

(52) U.S. Cl. 600/381

(57) ABSTRACT

An implantable device is described for detecting an expansion which is an elastic deformation of an intracorporeal vessel wall. The device comprises a support structure which contains dielectric polymer, has surface elasticity and can be applied directly or indirectly to the vessel wall, which provides at least one capacitive electrode arrangement, of which the assignable electrical capacitance can be influenced by an elastic deformation of the support structure. The electrode arrangement includes at least two electrodes each consisting of an electrically conductive polymer. The electrodes each define at least one side an intermediate space that influences the electrical capacitance of the electrode arrangement. The space is filled with the dielectric polymer of the support structure.

### CROSS REFERENCE TO RELATED APPLICATION

Reference is made to German Patent Application Serial No. 10 2010 010 348.9, filed on Mar. 5, 2010, entitled “Implantable Device for Detecting a Vessel Wall Expansion,” and also filed as PCT/EP2011/000974, filed Feb. 28, 2011, which applications are incorporated herein by reference in their entirety.

This application is a Continuation-In-Part of PCT/EP2011/000974.

### BACKGROUND OF THE INVENTION

1. Field of the Invention

The invention relates to an implantable device for detecting an expansion, that is an elastic deformation of an intracorporeal vessel wall, for example, of the stomach, the oesophagus, veins, arteries etc. comprising a support structure which contains a dielectric polymer, has surface elasticity and can be applied directly or indirectly to the vessel wall, which provides at least one capacitive electrode arrangement, having an assignable electrical capacitance which can be influenced by an elastic deformation of the support structure.

2. Description of the Prior Art

Implantable devices for detecting shape changes at vessel walls are used primarily for intracorporeal blood pressure measurement of blood-carrying vessels. For this purpose, predominantly flexural elastic sensors are known which operate on the basis of capacitive or resistive strain gauges and can be attached to the vessel outer wall without adversely effecting substantially the natural deformation of the vessel wall.

An implantable blood pressure sensor of this type is described in DE 10 2005 035 022 A1 which provides a means consisting of an elastic material, which surrounds the blood-carrying vessel in annular form, whose elasticity approximately corresponds to the intrinsic elasticity of the vessel wall so that the annular means does not permanently impart the natural deformation behavior of the vessel wall caused by the blood pressure. A strain gauge with capacitively or inductively acting electrode structures is attached to the annular means to measure the deformation behavior. In the case of capacitively acting electrode structures, the electrode structures configured as capacitor electrodes are located on opposite annular regions so that the capacitor electrodes influence the blood-carrying vessel on both sides like a dielectric.

A comparable sensor unit is described in EP 1 635 158 A2 which provides at least two capacitor electrodes inserted in a strip-shaped elastic carrier element to form a closed ring to detect pulsation expansion of a blood-carrying vessel. Plate spacing is determined by the diameter of the blood-carrying intracorporeal vessel. The arrangement of two capacitor electrodes, each opposite the blood-carrying vessel is quite essential here. As a result of the pulsing expansion of the blood vessel, the capacitor plate spacing and therefore the electrical capacitance of the measuring arrangement changes at the same time. In order to determine blood pressure and detect and transmit measurement data, a planar coil, connected to the capacitor electrodes, is additionally provided to form a resonator circuit whose resonance frequency depends on the electrical capacitance of the pressure-sensitive sensor. The signal which can be read out by the resonance circuit can be detected by a receiving unit provided extracorporeally, which additionally provides a measure of the shape change of the blood vessel and ultimately is a measure for the blood pressure.

Both of the above-described implantable blood pressure measuring systems each have capacitor electrode surfaces made of thin metal which preferably is copper or gold and which have none or only a limited expansibility. In order not to adversely affect the natural deformability of the blood vessel, it is therefore important to keep the electrode surfaces as small as possible which results in the signal levels which can be evaluated for the blood pressure detection being very small.

In addition, in the above-described implantable blood pressure measurement arrangements, the capacitor electrode surfaces are disposed opposite one another relative to the blood vessel. They therefore form an electrical capacitor whose plate spacing is defined by the diameter of the blood vessel. The interposed dielectric therefore comprises the tissue material of the blood vessel and the blood flow flowing through the blood vessel. As a result, the dielectric constant must be assumed to be non-constant as a result of the pulsing flow behavior. In addition, the blood is electrically conductive which results in electrical losses occurring in the capacitive measurement, which additionally adversely affect the measured signal level which can be evaluated. Another disadvantage as a result of the capacitor electrode surfaces being located opposite to one another on the vessel to be measured is the appearance of electrical scatter fields which occur as a result of relative movements of the blood vessel to be measured relative to the surrounding tissue which can also permanently adversely affect the measurement results.

### SUMMARY OF THE INVENTION

The invention is an implantable device for detecting an expansion, that is an elastic deformation of an intracorporeal vessel wall comprising a support structure which contains a dielectric polymer, has surface elasticity and can be applied directly or indirectly to the vessel wall, which provides at least one capacitive electrode arrangement having an assignable electrical capacitance which is influenced by an elastic deformation of the support structure in such a manner that on the one hand care should be taken not to influence the natural deformation behavior of the vessel wall. The signal quality and signal strength which can be evaluated is considerably improved. In particular, it is important to avoid falsely perturbing influence on the measured signals and ultimately on the measurement result, such as for example due to the occurrence of scatter fields or due to temporally varying dielectric properties of the dielectric disposed between the electrode surfaces etc.

In order to measure the natural pulsing deformation behavior of intracorporeal vessel walls composed of a very soft and highly flexible tissue material, that typically has an elastic modulus of 1 MPa and a ductility of 10% or more, without any influence that constricts and causes local surface stiffening of the elastically deforming vessel wall, a strain gauge is used with highly elastic and flexible materials, based on the capacitive measurement principle, which is not subject to any significant perturbing influences and also offers various embodiments.

The elastic deformation of the support structure is comparable to elasticity of the intracorporeal vessel wall. As a result, the support structure has no effect or only an insignificant effect on the expansion properties of the vessel wall and does not damage the viability of the vessel wall.

According to the invention, the implantable device for detecting an expansion from an elastic deformation of an intracorporeal vessel wall, comprises a support structure which contains a dielectric polymer, has surface elasticity and can be applied directly or indirectly to the vessel wall. The device provides at least one capacitive electrode arrangement with an assignable electrical capacitance which can be influenced by an elastic deformation of the support structure, in which the electrode arrangement provides at least two electrodes each composed of an electrically conductive polymer which limits at least one side of an intermediate space that influences the electrical capacitance of the electrode arrangement and is completely filled with the dielectric polymer of the support structure.

The term “vessel wall” is to be understood as an intracorporeal wall composed of biological tissue, such as for example, the vessel wall of arteries, veins, capillaries or lymph vessels and organ walls, such as the stomach wall, the oesophagus, intestinal wall or similar thereto are also applicable to the invention.

Unlike the known comparable implantable measuring devices, the device according to the invention provides an electrode arrangement preferably comprising completely an electrically conductive polymer which, as a result of its inherent surface elastic properties and its shape and size, has no effect or only an insignificant effect on the natural expansion properties of the vessel wall. In particular, the electrode arrangement is configured as capacitor electrodes with the dielectric determining the capacitance of the electrode arrangement being exclusively determined by the choice of the material of the support structure. As a result, it is already possible to calibrate the measuring device outside the body, that is “ex vivo”. Potential measurement errors caused by variable dielectric constants such as, for example, by a dynamic blood flow which flows between the capacitor electrodes can be intentionally avoided.

In order to explain the structure of an implantable device configured according to the invention and its advantageous further developments, reference is made to the following descriptions which refer to the specific exemplary embodiments shown in the figures.

### BRIEF DESCRIPTION OF THE DRAWINGS

The invention will be described as an example hereinafter without restricting the general inventive idea by means of exemplary embodiments with reference to the drawings. In the figures:

FIGS. 1a-d show multisided views of an exemplary embodiment configured according to the invention;

FIGS. 2a-d show an exemplary embodiment configured according to the invention with structured electrode arrangement;

FIGS. 3a-d show a multisided view of an exemplary embodiment configured according to the invention with interdigital electrode arrangement;

FIGS. 4a-d show a multisided view of an extension variant to FIG. 3;

FIG. 5 shows a cross-section through an exemplary embodiment with additional shielding;

FIG. 6 shows a further variant with external shielding; and

FIG. 7 shows an exemplary embodiment with shielding electrodes.

### DETAILED DESCRIPTION OF THE INVENTION

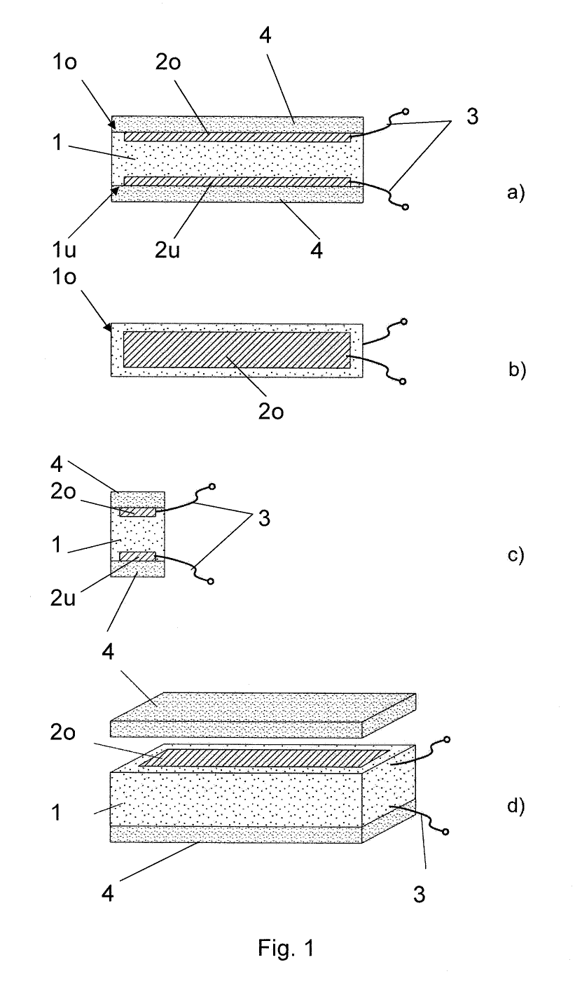

FIGS. 1a-d show the basic principle of the structure of an implantable device configured according to the invention in a multisided view. FIG. 1a shows a longitudinal section through a device configured according to the invention, which provides a support structure 1 composed of a dielectric polymer which preferably is a highly elastic silicone, that has an intrinsic elasticity which is comparable with the elasticity of the intracorporeal wall. That is the polymer has an elastic modulus of about 1 MPa. Such polymer materials are known in the art and can be produced industrially. The support structure 1 is configured to be flat and has a support structure upper side 1o and a support structure lower side 1u. Typically the support structure has a thickness of several 10 s to several 100 s μm, for example, 50 μm to 400 μm. Electrodes 2o and 2u, respectively each composed of an electrically conductive polymer layer, are mounted on the structure upper side 1o and on the structure lower side 1u. Both electrodes 2o and 2u are connected to electrical contacts 3 which for example are in the form of thin wires. In the exemplary embodiment according to FIG. 1, the electrodes 2o and 2u are flat, strip-shaped and each embedded on the support structure upper side 10 and the support structure lower side 1u. That is, the electrodes are each surrounded laterally by a circumferential support structure collar. FIG. 1b shows in a sectional view the plan view of the support structure upper side 1o with the upper electrode layer 2o embedded therein.

For the purpose of electrical insulation and encapsulation of the electrodes 2o and 2u are composed of an electrically conductive polymer and one insulation layer 4 is applied at least on the upper and lower side of the support structure 1, which can comprise the same surface-elastic dielectric polymer of the support structure or of a polymer material different from the support structure which however has comparable elasticity properties. Merely for reasons of better illustration, the upper insulation layer 4 has been omitted in the plan view according to FIG. 1b.

The cross-sectional view according to FIG. 1c illustrates the application of the upper and lower electrically conductive polymer electrode layers 2o and 2u which are each surrounded laterally by the support structure 1. The electrodes 2o and 2u enclose a mutual spacing which is completely filled with the highly elastic polymer of the support structure 1. Thus, exclusively the polymer material of the support structure is the dielectric of the capacitance of the electrode arrangement. As a result of a mechanical deformation of the electrode arrangement illustrated in FIG. 1 together with support structure and the insulating layers 4 applied thereon in each case, a change in the electrode spacing between the two conductive polymer layers occurs, which results in a measurable change in the capacitance. For the capacitance measurement, an electrical potential difference is applied between the two electrodes 2o and 2u, which can be applied by the two contact wires 3.

One way of reading out the sensor is to connect the contact wires 3 to an implantable electrical read-out unit having an integrated energy source which, for example, may be a battery or providing the energy supply by providing an extracorporeal wireless energy supply technique. For this purpose, the electrodes 2o and 2u should be connected via the contact wires 3 to a suitable inductance which is implemented or embedded like the electrodes 2o and 2u inside the support structure 1. With the aid of an extracorporeally provided coil, electrical energy can be coupled by inductive coupling into the inductance provided intracorporeally inside the support structure 1.

The capacitance change caused by deformation can be detected with an extracorporeally provided energy supply unit which is typically an antenna unit. The inductance inside the support structure and connected to the capacitive electrodes 2o and 2u forms an oscillatory circuit, whose resonance frequency is substantially determined by the capacitance of the capacitive electrode arrangement. A change in capacitance is reflected in a change in the resonance frequency of the oscillatory circuit which in turn can be detected with the extracorporeally provided antenna unit. Such wireless detection of a measured signal is known in the art as GRID dipping.

FIG. 1d shows a perspective view of the implantable device configured according to the invention in which the upper insulator layer 4 is shown slightly raised to make the upper electrode 2o visible. The layer composite shown only in sections in FIG. 1 comprising the insulator layers 4, the electrode layers 2o and 2u and the interposed support structure 1 is configured, for example, as longitudinal strips having a strip length which can be laid once flush around the external circumference of a vessel carrying blood. For fastening the strip-shaped layer composite on the intracorporeal vessel wall, preferably two strip region ends are joined firmly to one another by gluing, welding, clamping or sewing techniques.

The blood pressure sensor illustrated in FIG. 1 is preferably accomplished in layer form, by applying a largely unstructured, preferably rectangular electrode surface 2u to a first lower dielectric layer 4. An electrically conductive polymer, for example, in a flowable state or in a prefabricated solid film state, is applied to the surface of the dielectric 4. Subsequently, the electrically insulating dielectric of the support structure is applied, where the lower electrode surface 2u is completely potted or enclosed by the support structure material both laterally and on its free surface. Finally, the upper electrode 2o and the dielectric layer 4 are applied. As has already been explained previously, the distance between the two electrode layers 2o and 2u, which is the thickness of the dielectric inside the support structure 1, may be between 50 μm to 400 μm with a preferred thickness of 100 μm. The thicknesses of the electrode layers 2o and 2u are chosen to be approximately the same and thinner than the thickness of the support structure 1. Advantageously both electrodes 2o and 2u are arranged congruently vertically one each other in order to achieve a maximum degree of overlap. Equally, however it is also possible to arrange the electrodes 2o and 2u in a suitable manner offset to one another or to provide additional contact elements in order to be able to make possible fine tuning of the resonance circuit.

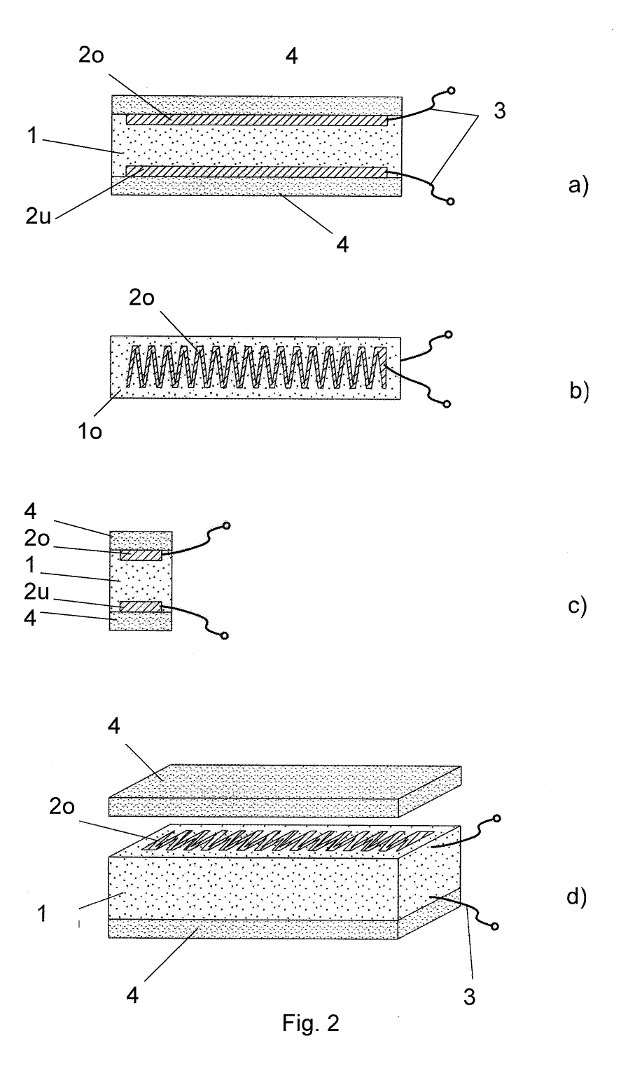

A second embodiment illustrated in FIGS. 2a-d of an implantable device according to the invention fundamentally has the same components as the exemplary embodiment illustrated in FIG. 1 [-] with identical reference numbers being used to identify the same components which have been already described, In this case, the electrode surfaces 2o and 2u are not configured in the form of unstructured rectangular surface electrodes but instead are structured in the form, such as for example, a zigzag pattern (See FIG. 2b). Due to the zigzag structure of the upper and of the lower electrode 2o and 2u, a substantially higher surface elasticity of the capacitive electrodes 2o and 2u is achieved as compared with the previously described, unstructured embodiment of the electrodes 2o and 2u in FIG. 1. Advantageously the structured electrodes 2o and 2u in the form of an orthogonal projection onto the support structure upper and lower side 1o and 1u are arranged as congruently as possible. As in the case of the exemplary embodiment in FIG. 1, a dielectric polymer, which is preferably an elastic stretchable silicone, providing the dielectric of the capacitive electrode arrangement, is located between structured electrodes 2o and 2u.

The structure of the electrodes 2o and 2u can be achieved by laser processing. For example, the laser processing can provide an unstructured rectangular electrically conductive polymer layers which are already applied to the support structure. The laser beam is absorbed by the material of the electrically conductive polymer. The laser can penetrate largely without absorption through the dielectric support structure material. With the laser processing method, in addition to approximately arbitrary two-dimensional structures can be processed from a homogeneous application, an electrically conductive polymer layer is additionally possible to shape the boundary edges of the structured electrode layer in relation to the steepness of the flank. In this way, the electrical fields advantageously formed between the two electrodes separated by the dielectric of the support structure can advantageously be configured or optimized to avoid perturbing scatter fields.

In addition to an improved ductility, structured electrodes, formed preferably in the manner described in FIGS. 2b and d, avoid fractures or cracking in cases of large deformation and therefore help the sensor to have a greater robustness and longer lifetime.

In the two previously described embodiments, the electrodes 2o and 2u composed of an electrically conductive polymer are located in two different parallel planes inside the support structure 1, Subsequent embodiments are described in which both electrodes are disposed in a single plane and therefore provide an implantable device having significantly reduced thickness.

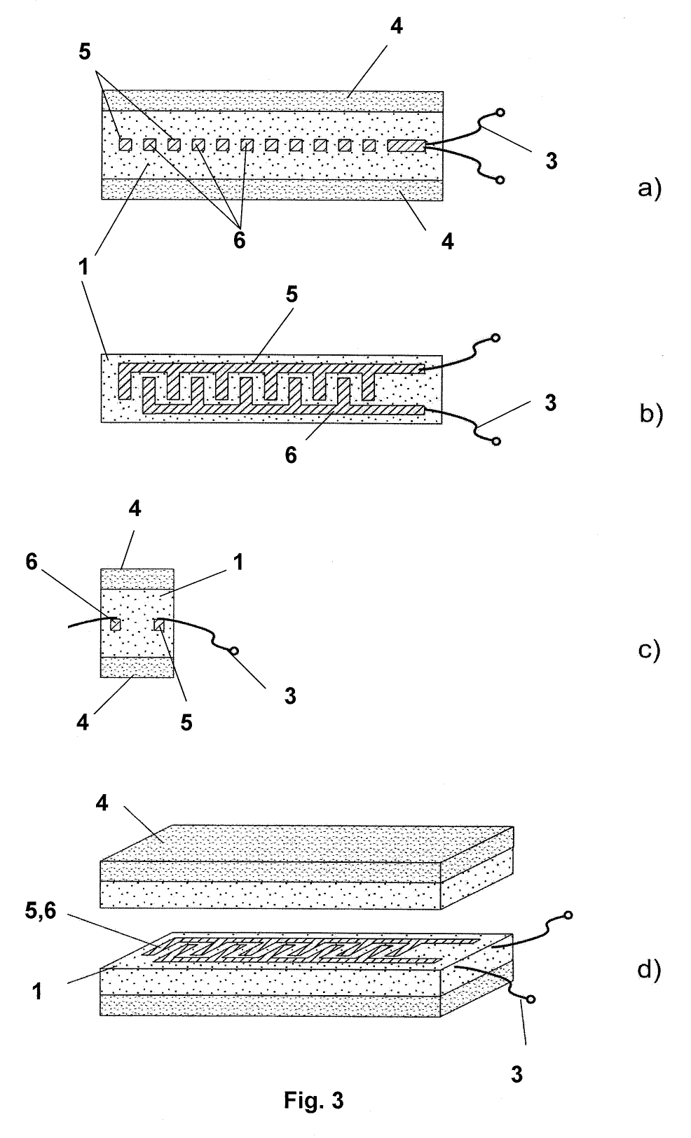

FIGS. 3a to 3d show a multisided view of an implantable device in the same way as FIGS. 1 and 2, in which the electrodes are composed of an electric polymer are configured as interdigital structure electrodes. This is described in particular in a plan view according to FIG. 3b which shows a central plane of intersection through the support structure 1 in which two electrodes 5 and 6, configured as interdigital electrodes, are either disposed or embedded. FIG. 3a shows a corresponding longitudinal section through the implantable device with insulator layers 4 applied to the support structure upper and lower side 1o and 1u. FIG. 3c shows a corresponding cross-section through the device. FIG. 3d illustrates a perspective overall view of the implantable device having a layered structure, in which the layer structure is shown in two vertically spaced-apart halves to provide visualization of the interdigital electrode arrangement 5 and 6.

The electrodes 5 and 6 are an electrically conductive polymer which is configured in the form of an interdigital structure. Each electrode comprises finger structures which mutually intermesh in a contactless manner within one plane. The space between the interdigital electrode structures, each lying opposite one another in the plane, is completely filled with the highly elastic polymer support structure material, which is preferably silicone, is the dielectric between the electrodes. When applying an electrical potential difference between both electrode structures 5 and 6, electrical field lines are formed which are perpendicular to the electrode surface between the immediately opposite electrode planes. In addition, elliptical electrical field lines are formed between the finger electrode structures running above and below the electrode planes 5 and 6 in the support structure 1. The elliptical field lines provide a substantial contribution to the measurable capacitance. A mechanical deformation of the sensor illustrated in FIG. 3 results in a change in the spacing between the individual electrode structure fingers of the interdigital electrode structure, which causes the capacitance of the electrode arrangement to vary in an extremely sensitive manner, which can be measured.

The interdigital electrode arrangements of FIGS. 3b and 3d should be considered as a stylized schematic reproduction of a classical interdigital electrode structure. With the aid of the previously described laser processing already discussed, very delicate interdigital electrode structures can be formed from flat deposited polymer layers which are configured to be optimized with respect of their elasticity. One such embodiment of an elasticity-optimized electrode structures are horseshoe-shaped conductor tracks formed by arranging pairs of horseshoes curved by 120°.

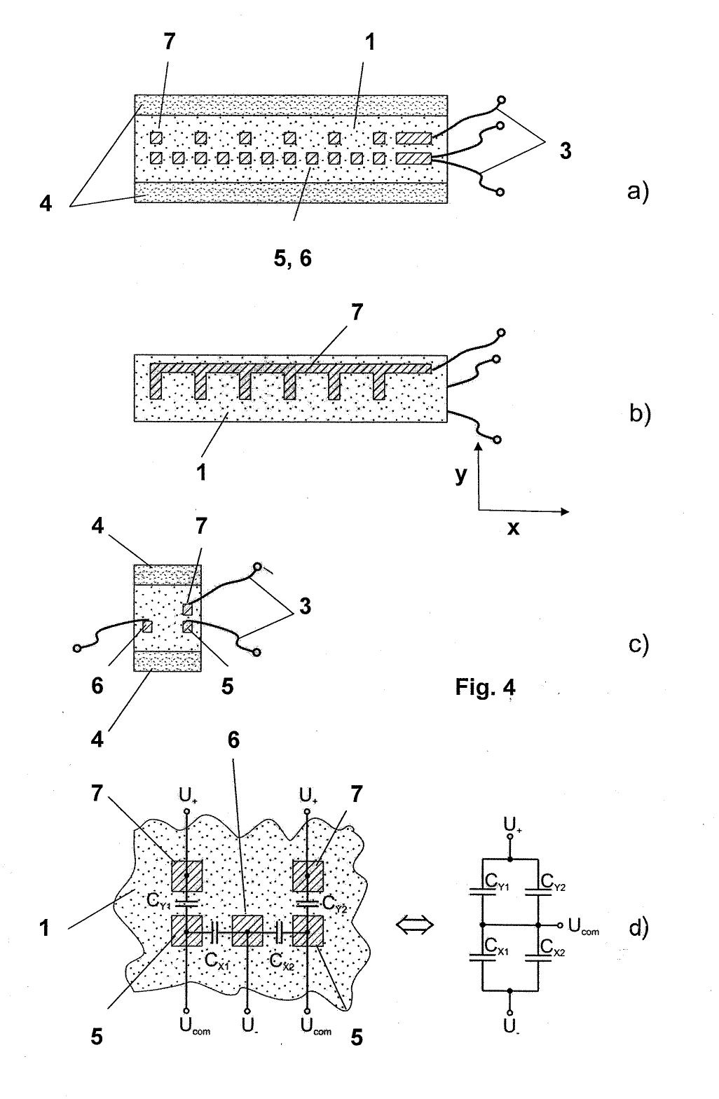

Another embodiment for an implantable device having an interdigital electrode structure arrangement is shown in FIGS. 4a-d, which compared to the exemplary embodiment according to FIG. 3, has another interdigital electrode 7 disposed in a second plane within the support structure 1 which is oriented parallel to the first plane in which the structure arrangement 5 and 6 illustrated in FIG. 3 is placed. In particular, the further interdigital electrode structure 7 is arranged parallel to the interdigital electrode structure 5. Like the preceding figures, FIG. 4a shows a longitudinal sectional view, FIG. 4b shows a plan view and FIG. 4c shows a cross-sectional view through the electrode arrangement.

As a result of the additional interdigital electrode structure 7 provided in the second plane, a so-called differential capacitor is achieved. With the design of the differential capacitor, perturbations and in particular all common mode perturbations such as, for example, parasitic capacitances, offset errors, operating voltage and temperature influences, can be effectively suppressed. In addition, the sensitivity of the sensor is increased for deformations both in the longitudinal, x direction and in the spatial direction y oriented orthogonally to the longitudinal direction. The principle of a differential capacitor is explained by the equivalent circuit diagram to FIG. 4d. The left-hand diagram in FIG. 4d shows a section of a longitudinal section according to FIG. 4a in which the interdigital electrode arrangement 5 and 6 and the interdigital electrode structure 7 arranged separately in the second plane are seen. It is thus assumed that the support structure 1 in the left-hand diagram according to FIG. 4d experiences a stretching in the x direction with the result that the distances between the interdigital electrodes 5 and 6 are increased which results in the capacitances Cₓ₁ and Cₓ₂ each being reduced. At the same time, the support structure 1 experiences a compression in the y direction due to a transverse contraction, with the result that the distances between the interdigital electrode structures 5 and 7 becomes smaller and therefore the capacitances C_{y1} and C_{y2} increase accordingly in the opposite direction to Cₓ₁ and Cₓ₂. With a corresponding design, the capacitances and the changes in capacitance can have the same dimensions. With the aid of the equivalent circuit diagram illustrated in the right-hand diagram according to FIG. 4d, the selectivity of the sensor in this measurement mode is increased for changes in deformation both in the x and in the y direction. In addition, an effective suppression of interference is achieved.

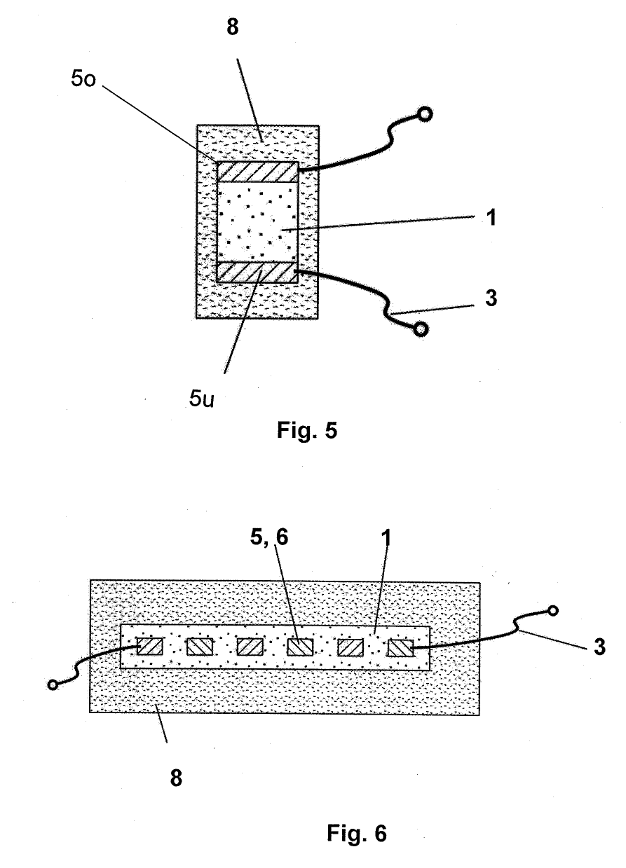

Depending on their design structure, capacitive sensors fundamentally generate electromagnetic scatter fields which, depending on the manifestation, can extend into the spatial surroundings of the capacitor arrangement. If the electrical or dielectric properties of the surroundings change, the scatter fields active in the surroundings can ultimately have an effect on the capacitance of the sensor and influence this. If it is necessary to make capacitive measurements with only low signal levels, as is the case in the apparatus according to the invention, it is desirable to suppress any perturbing sources of error which influence the measurement results. In particular, it is necessary to minimize the falsification of the sensor signals caused by scatter fields and their environment-dependent feedback on the measurement signal. One possibility for reducing this perturbing effect is specific focusing of the electric field lines formed between the electrode structures on the area between the electrodes. It is known that electrical field lines are “guided” better in a dielectric material having a high dielectric constant than in a material having lower dielectric constant. This effect is used in a preferred exemplary embodiment of the implantable device according to the invention whereby the electrode structures with the dielectric material of the support structure inserted between the electrodes are completely surrounded by a material having a higher dielectric constant than the material in the further surroundings. In this way, the electric field is localized in a defined space around the electrode arrangement whereby the influence of possible scatter effects interacting with the surroundings is reduced. In order to influence a preferred exemplary embodiment in this respect, reference is made to FIG. 5 which shows a cross-section in the device configured according to the invention. Two electrodes 2o and 2u are mounted on the support structure upper side and on the support structure lower side as in the exemplary embodiment in FIG. 1 which is additionally surrounded with a highly elastic dielectric polymer layer 8, whose assignable dielectric constant is lower than the dielectric constant of the dielectric polymer of the support structure 1. A device with interdigital electrode structure according to FIG. 6 can also have a comparable encasing which is completely surrounded by an insulator layer 8 whose dielectric constant is lower than the dielectric constant of the dielectric of the support structure 1.

In addition, the propagation of scatter field can be reduced by a suitable adaptation of the structural quantities of the individual components of the device. If, for example, the electrode spacing between two electrodes is small compared with its surface or lateral expansion, the electrical field remains substantially focused within the capacitor structure as a result of the small electrode spacing and cannot be scattered into the surroundings.

Another possibility for reducing perturbing influences is to completely or partially externally surround the electrode structures with extra shielding electrodes. With the aid of these externally applied shielding electrodes which are placed at a defined electric potential, the scatter fields emanating from the capacitive electrodes are regularly prevented from “penetrating” into the spatial area surrounding the sensor unit. In the same way, external perturbing fields cannot “penetrate” from outside into the capacitive sensor structure.

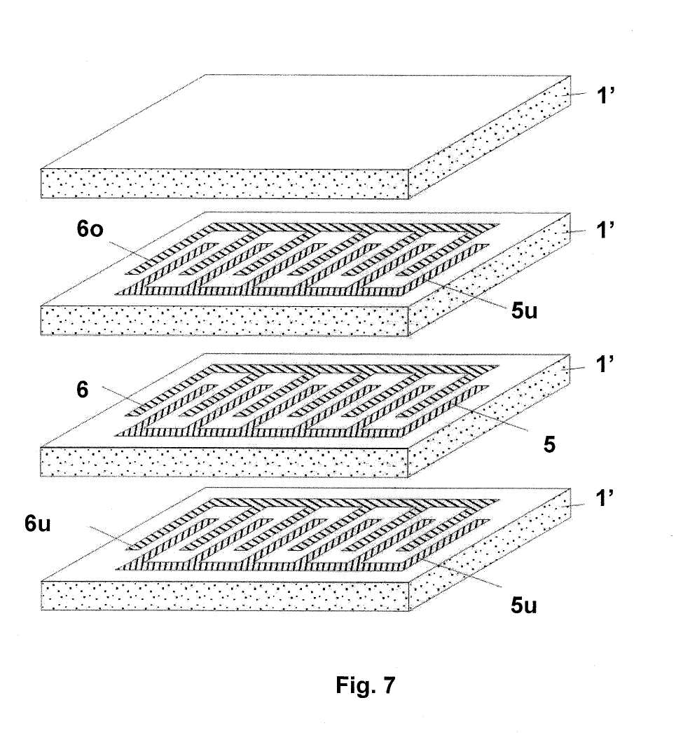

In order to not significantly impair the intrinsic elasticity of the sensor device, shielding electrodes are only provided locally where a possible scatter field emission from the electrode structures is greatest. The shielding electrodes are preferably designed in the form of a grid structure so that the elastic deformability of the entire sensor device is substantially preserved. In the case of capacitive interdigital electrode structures, as is the case in the exemplary embodiment according to FIGS. 3 and 4, the shielding electrodes can be structured in the same form as the interdigital electrodes themselves. Such an exemplary embodiment is illustrated in FIG. 7 which, for purposes of better illustration, is shown in layers which are to be combined.

Thus, each interdigital electrode 5 and 6 is provided with its own shielding electrode 5o, 5u, 6o and 6u which has the same basic form as the interdigital electrode 5 and 6 itself and is separated by an insulating support structure intermediate layer 1′ which is mounted above or below layer 1′. As a result, the scatter capacity between the interdigital electrodes 5 and 6 and the shielding electrodes 5o, 5u, 6o and 6u is at least largely minimized in particular when the potential of each shielding electrodes 5u, 5o, 6u and 6o is tracked to the potential of the proximate interdigital electrodes 5 and 6.

Naturally, it is also possible to configure and arrange the shielding electrodes differently from the shape and size of the capacitive electrode arrangement.

Another embodiment which is not further illustrated, is also based on an interdigital structure which, however, is distinguished by a particularly high aspect ratio of the finger structures. Here the capacitance to be measured is principally composed of the parallel electrical field formed between two opposite electrode structures. From the production technology viewpoint, such a capacitive strain gauge can be designed in such a manner that a strip of highly elastic silicone material is coated on both sides with the electrically conductive polymer. The coating can be accomplished for example by a doctor blade technique or by evaporation. The coated strip is then rolled up in meander form and potted with the highly elastic silicone material. By contacting the conductive layers and applying a potential difference, a capacitance can also be detected here. As a result of mechanical deformation, a change in the space between the conductive layers occurs in this arrangement. This spatial difference results in a change in the capacitance which is a measurable quantity.

As already mentioned, the capacitive sensor principle according to the invention allows a wireless read out of the electrically measured signal by the known “grid dipping” principle. In this case, the sensor capacity of the electrode arrangement is connected to an electrical inductance to form an electrical oscillatory circuit. The inductance is constructively configured so that an electromagnetic alternating field that is emitted from outside by a transmitter can induce an electrical alternating current in the oscillatory circuit. If the frequency of the external alternating field corresponds to the electrical resonance frequency of the oscillatory circuit, the oscillatory circuit resonates and extracts a maximum of field energy from the external alternating field. This reduction in the field energy can be detected by the transmitter of the external alternating field. If the external transmitter now transmits an alternating field having time-variable frequency, it will then detect a selective interruption of the field energy at a resonance frequency of the oscillatory circuit. This allows the resonance frequency to be determined from outside. The resonance frequency of an oscillatory circuit comprising the capacitive electrode arrangement and the inductance depends, according to known physical laws, on the constant inductance of the coil and the variable capacitance of the electrode arrangement. Thus, the capacitance can be detected in a wireless manner via the detected variable resonance frequency.

In a practical embodiment, the inductance is integrated directly in the support structure. The connection to the sensor capacitance is also made inside the support structure. In this way, all electrical lines inside the support structure can be protected. An electrical feed-through of lines towards the outside is no longer necessary. In addition, the sensor signal can be read out in a wireless manner over a specific distance.

### REFERENCE LIST

1 Support structure
2 Electrode arrangement
2o Upper electrode
2u Lower electrode
3 Contact wire
4 Insulator layer
5 and 6 Electrodes
5u, 5o, 6u and 6o Shielding electrodes
7 Additional interdigital electrode
8 Shielding layer

1-14. (canceled)

15. An implantable device for detecting an expansion of an intracorporeal vessel wall, comprising:
a support structure containing a dielectric polymer having surface elasticity and being applied directly or indirectly to the vessel wall, at least one capacitive electrode arrangement of variable electrical capacitance influenced by an elastic deformation of the support structure, and an elasticity comparable to elasticity of the intracorporeal vessel wall; and wherein
the electrode arrangement includes at least two electrodes each comprising an electrically conductive polymer which contacts at least one side of an intermediate space filled with the dielectric polymer that influences the electrical capacitance of the electrode arrangement, the support structure being flat and has an upper and lower side, the electrodes each comprising an electrically conductive polymer layer and a different one of the layers being applied respectively to the upper and lower sides.

16. The device according to claim 15, wherein:
the dielectric polymer comprises elastic silicone.

17. The device according to claim 15, wherein:
the electrically conductive layers have a thickness that is less than or equal to a thickness of the support by which electrically conductive polymer layers are separated from one another.

18. The device according to claim 16, wherein:
the electrically conductive layers have a thickness that is less than or equal to a thickness of the support by which electrically conductive polymer layers are separated from one another.

19. The device according to claim 15, wherein:
the electrically conductive polymer layers are disposed to project to at least partially overlap orthogonally relative to the upper and lower side and are related in form to each other.

20. The device according to claim 16, wherein:
the electrically conductive polymer layers are disposed to project to at least partially overlap orthogonally relative to the upper and lower side and are related in form to each other.

21. The device according to claim 17, wherein:
the electrically conductive polymer layers are disposed to project to at least partially overlap orthogonally relative to the upper and lower side and are related in form to each other.

22. The device according to claim 18, wherein:
the electrically conductive polymer layers are disposed to project to at least partially overlap orthogonally relative to the upper and lower side and are related in form to each other.

23. The device according to claim 15, wherein:
the electrically conductive polymer layers of the upper and lower side are each covered with an elastic electrical insulator.

24. The device according to claim 16, wherein:
the electrically conductive polymer layers of the upper and lower side are each covered with an elastic electrical insulator.

25. The device according to claim 17, wherein:
the electrically conductive polymer layers of the upper and lower side are each covered with an elastic electrical insulator.

26. The device according to claim 18, wherein:
the electrically conductive polymer layers of the upper and lower side are each covered with an elastic electrical insulator.

27. The device according to claim 19, wherein:
the electrically conductive polymer layers of the upper and lower side are each covered with an elastic electrical insulator.

28. The device according to claim 20, wherein:
the electrically conductive polymer layers of the upper and lower side are each covered with an elastic electrical insulator.

29. The device according to claim 21, wherein:
the electrically conductive polymer layers of the upper and lower side are each covered with an elastic electrical insulator.

30. The device according to claim 22, wherein:
the electrically conductive polymer layers of the upper and lower side are each covered with an elastic electrical insulator.

31. An implantable device for detecting an expansion of an intracorporeal vessel wall, comprising:
a support structure containing a dielectric polymer having surface elasticity and being applied directly or indirectly to the vessel wall, at least one capacitive electrode arrangement of variable electrical capacitance influenced by an elastic deformation of the support structure, and an elasticity comparable to elasticity of the intracorporeal vessel wall; and wherein
the electrode arrangement includes at least two electrodes each comprising an electrically conductive polymer which contacts at least one side of an intermediate space filled with the dielectric polymer that influences the electrical capacitance of the electrode arrangement, the support structure being flat and has an upper and lower side, the electrodes each comprising an electrically conductive polymer layer and a different one of the layers being applied respectively to the upper and lower sides; and
that the at least two electrodes are disposed in a first plane extending between the upper and lower side, being oriented parallel to the upper and lower side, being spaced apart laterally to one another in the plane and being surrounded by the dielectric polymer of the support structure.

32. The device according to claim 31, wherein:
the electrodes each have an elongate electrode section from which electrode fingers branch off orthogonally to provide a longitudinal extension thereof and two finger electrodes immediately adjacent each other along the elongate electrode section define a U-shaped intermediate space into which one finger electrode of another electrode projects.

33. The device according to claim 31, wherein:
a second plane extends between the upper and lower side and is oriented parallel to and spaced from the first plane, at least one further electrode is located in the first plane and projects orthogonally relative to the planes to at least partially overlap the electrode located in the first plane.

34. The device according to claim 15, wherein:
the at least two electrodes are each connected to an electrical contact to which at least one inductance is connected to form an oscillatory circuit.

35. The device according to claim 31, wherein:
the at least two electrodes are each connected to an electrical contact to which at least one inductance is connected to form an oscillatory circuit.

36. The device according to claim 15, wherein:
the support is at least partially surrounded by a dielectric polymer material having a dielectric constant lower than a dielectric constant of the dielectric polymer.

37. The device according to claim 31, wherein:
the support is at least partially surrounded by a dielectric polymer material having a dielectric constant lower than a dielectric constant of the dielectric polymer.

38. The device according to claim 15, comprising:
a shielding electrode associated with each electrode;
a dielectric electrode disposed between the shielding electrode and an electrode associated with the shielding electrode; and
the shielding electrode is disposed on a side facing away from the electrode arrangement relative to the electrode associated with the shielding electrode.

39. The device according to claim 31, comprising:
a shielding electrode associated with each electrode;
a dielectric electrode disposed between the shielding electrode and an electrode associated with the shielding electrode; and
the shielding electrode is disposed on a side facing away from the electrode arrangement relative to the electrode associated with the shielding electrode.

40. The device according to claim 38, wherein:
the shielding electrode comprises an electrically conductive polymer layer which is contacted electrically by an electrical connector

41. The device according to claim 39, wherein:
the shielding electrode comprises an electrically conductive polymer layer which is contacted electrically by an electrical connector.
